(19) United States
(12) Patent Application Publication
(10) Pub. No.: US 2026/0115017 A1
(43) Pub. Date: Apr. 30, 2026

(54) BREAST PROSTHESIS AND METHOD FOR MANUFACTURING THE SAME

(71) Applicant: The Hong Kong Polytechnic University, Hong Kong (CN)

(72) Inventors: Ruixin Liang, Hong Kong (CN); Yiu-Wan Joanne Yip, Hong Kong (CN); Lin Zheng, Hong Kong (CN)

(73) Assignee: The Hong Kong Polytechnic University, Hong Kong (CN)

(21) Appl. No.: 19/351,555

(22) Filed: Oct. 7, 2025

(30) Foreign Application Priority Data
Oct. 24, 2024 (CN) 2024114921513

(51) Int. Cl. A61F 2/52; A61F 2/50; A61F 2/74; A61L 27/18; B33Y 50/00; B33Y 80/00; D06H 5/00

(52) U.S. Cl. CPC A61F 2/52; A61F 2/5044; A61F 2/748; A61L 27/18; B33Y 50/00; B33Y 80/00; D06H 5/00; A61F 2002/501; A61L 2430/04; D10B 2331/10; D10B 2509/00

(57) ABSTRACT

The present disclosure provides a breast prosthesis and a method for manufacturing the same, which relate to the field of prosthesis manufacturing technologies. The breast prosthesis includes an outer shell and an airbag inner layer. A cavity is formed inside the outer shell, and the outer shell is provided with an opening in communication with the cavity. The airbag inner layer is provided with a gas valve.

### CROSS REFERENCE

The present application is based on and claims priority to Chinese Patent Application No. 2024114921513, filed on Oct. 24, 2024, the entire contents of which are incorporated herein by reference.

### TECHNICAL FIELD

The present disclosure relates to the field of prosthesis manufacturing technologies, and in particular to a breast prosthesis and a method for manufacturing the same.

### BACKGROUND

Breast cancer stands as the most common cancer among women globally, with over 2 million new cases reported in 2020 according to the Global Cancer Observatory. Surgery is a primary treatment for about 90% of women diagnosed with breast cancer. This can range from a lumpectomy, which aims to remove the tumor while preserving most of the breast, to a mastectomy, which involves removing the entire breast and is necessary for 28% to 60% of patients, a figure that is increasing.

Despite the option for breast reconstruction after surgery, many women opt against it, turning instead to external breast prostheses. These prostheses are designed to replicate the natural appearance and feel of the breast and come in various types to meet the diverse needs of women post-mastectomy.

The diverse selection of materials and methods for attaching breast prostheses enables a high degree of personalization in post-mastectomy care. Given that preferences and needs differ significantly among individuals, the advancement of prosthetic development must prioritize customizable solutions. Such an approach is essential to address the unique needs of each woman effectively, thereby improving their quality of life and self-assurance following surgery.

It should be noted that the information disclosed in the Background section above is only for enhancing the understanding of the background of the present disclosure, and thus may include information that does not constitute prior art known to those of ordinary skill in the art.

### SUMMARY

The present disclosure is to provide a breast prosthesis and a method for manufacturing the same.

According to an aspect of the present disclosure, there is provided a method for manufacturing a breast prosthesis. The breast prosthesis includes an outer shell and an airbag inner layer. A cavity is formed inside the outer shell, and the outer shell is provided with an opening that is in communication with the cavity. The airbag inner layer is provided with a gas valve. The method includes: manufacturing the outer shell using a first mold and a second mold, wherein the second mold is enclosed by the outer shell, and the second mold is made of a soluble material; demolding the outer shell and immersing the outer shell in a solution to dissolve the second mold within the outer shell to form the cavity inside the outer shell; forming the opening in the outer shell; manufacturing the airbag inner layer; and installing the gas valve on the airbag inner layer to locate the gas valve at the opening of the outer shell upon placing the airbag inner layer into the cavity of the outer shell through the opening.

According to another aspect of the present disclosure, there is provided a breast prosthesis, which includes: an outer shell, wherein a cavity is formed inside the outer shell, the outer shell is provided with an opening, and the opening is in communication with the cavity; and an airbag inner layer located within the cavity, wherein the airbag inner layer is provided with a gas valve for filling the airbag inner layer with gas.

It should be noted that the above general description and the following detailed description are merely exemplary and explanatory and should not be construed as limiting of the disclosure.

### BRIEF DESCRIPTION OF THE DRAWINGS

The drawings herein are incorporated in and constitute a part of the specification, illustrate embodiments consistent with the present disclosure, and together with the description serve to explain principles of the present disclosure. Apparently, the drawings in the following description are only some embodiments of the present disclosure. For those of ordinary skill in the art, other drawings may be obtained based on these drawings without paying any creative effort.

FIG. 1 shows a flowchart of a method for manufacturing a breast prosthesis according to an embodiment of the present disclosure;

FIG. 2 shows a flowchart of a method for manufacturing a silicone outer shell according to an embodiment of the present disclosure;

FIG. 3 shows a flowchart of a method for preparing a silicone material according to an embodiment of the present disclosure;

FIG. 4 shows a flowchart of a method for determining a mold using 3D scanning according to an embodiment of the present disclosure;

FIG. 5 shows a breast model extracted from a scanned model according to an embodiment of the present disclosure;

FIG. 6 shows a flowchart of a method for manufacturing an airbag inner layer according to an embodiment of the present disclosure;

FIG. 7A shows a bottom schematic diagram of a design pattern of an airbag (an example of a cup size 38C) according to an embodiment of the present disclosure;

FIG. 7B shows a first side schematic diagram of a design pattern of an airbag (an example of a cup size 38C) according to an embodiment of the present disclosure;

FIG. 7C shows a second side schematic diagram of a design pattern of an airbag (an example of a cup size 38C) according to an embodiment of the present disclosure;

FIG. 8A shows a schematic perspective view of a design of an airbag inner layer according to an embodiment of the present disclosure;

FIG. 8B shows a schematic diagram of a one-way gas valve according to an embodiment of the present disclosure;

FIG. 9 shows a schematic structural diagram of a breast prosthesis according to an embodiment of the present disclosure;

FIG. 10 shows a schematic diagram of breast prostheses with varying silicone weights according to an embodiment of the present disclosure;

FIGS. 11A to 11C show diagrams of a first portion of a first mold and a second mold at different angles according to an embodiment of the present disclosure;

FIGS. 12A to 12C show diagrams of a second portion of a first mold at different angles according to an embodiment of the present disclosure;

FIG. 13 shows top views of a first portion of a first mold, a second mold, and a second portion of the first mold according to an embodiment of the present disclosure;

FIG. 14 shows a perspective view of a first mold and a second mold after being combined according to an embodiment of the present disclosure;

FIG. 15 shows a side view of a first mold and a second mold after being combined according to an embodiment of the present disclosure;

FIG. 16 shows a schematic diagram of a silicone outer shell after demoulding according to an embodiment of the present disclosure;

FIG. 17 shows a schematic diagram of a silicone outer shell after forming an internal cavity according to an embodiment of the present disclosure; and

FIGS. 18A to 18B show schematic diagrams of an airbag inner layer at different angles according to an embodiment of the present disclosure.

### DETAILED DESCRIPTION

Example embodiments will now be described more fully with reference to the accompanying drawings. However, the example embodiments can be implemented in a variety of forms and should not be construed as being limited to examples set forth herein; rather, these embodiments are provided so that the present disclosure will be more complete and comprehensive so as to convey the idea of the example embodiments to those skilled in this art. The described features, structures or characteristics may be combined in any suitable manner in one or more embodiments.

In addition, the drawings are merely schematic representations of the present disclosure and are not necessarily drawn to scale. The same reference numerals in the drawings denote the same or similar parts, and the repeated description thereof will be omitted. Some of the block diagrams shown in the figures are functional entities and do not necessarily correspond to physically or logically separate entities. These functional entities may be implemented in software, or implemented in one or more hardware modules or integrated circuits, or implemented in different networks and/or processor devices and/or microcontroller devices.

At present, some women were not satisfied with the existing breast prostheses. A study in the year of 2023 highlighted that woman with external breast prostheses reported lower quality of life scores compared to those with immediate breast reconstruction. Dissatisfaction factors were ranked in order of dissatisfaction from high to low as follows: weight, comfort, movement with the body, fit, value for money, shape, temperature, appearance when worn, texture, durability, color, and quality.

The technical solution of a pneumatic breast prosthesis provided in the present disclosure uses an outer shell (e.g., a silicone outer shell) and an airbag inner layer. The airbag inner layer is located in a cavity of the outer shell and can be filled with gas. Through the cooperation of the outer shell and the airbag inner layer, the weight and shape of a non-surgical breast can be closely mimicked, thereby improving the patient's comfort and body balance after mastectomy.

In the breast prosthesis and the method for manufacturing the same provided by embodiments of the present disclosure, the design of the airbag inner layer in the outer shell reduces the weight of the breast prosthesis, solving the issue of discomfort caused by excessive heaviness.

Furthermore, by combining 3D scanning and 3D printing technologies, the breast prosthesis is matched to the non-surgical breast, resolving the issue of the breast prosthesis not fitting the body properly.

In some instances, the breast prosthesis may be called a prosthetic breast.

Hereinafter, each step of a method for manufacturing a breast prosthesis in embodiments of the present disclosure will be described in more detail with reference to the accompanying drawings and examples.

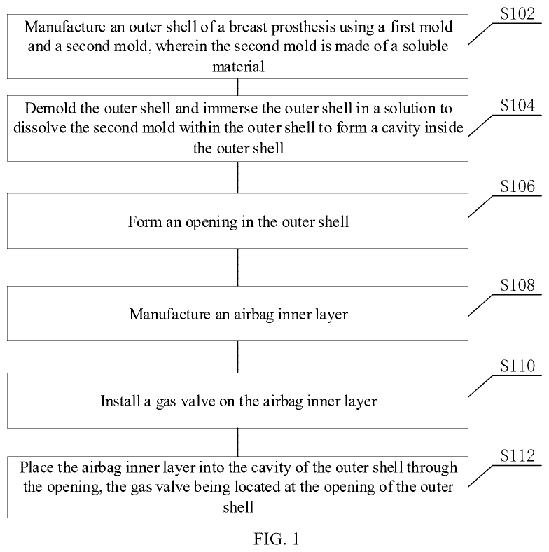

FIG. 1 shows a flowchart of a method for manufacturing a breast prosthesis according to an embodiment of the present disclosure. In this embodiment, the breast prosthesis includes an outer shell and an airbag inner layer. A cavity is formed inside the outer shell, and the outer shell is provided with an opening that is in communication with the cavity. The opening is used for inflation and deflation. The airbag inner layer is provided with a gas valve.

As shown in FIG. 1, in S102, the outer shell of the breast prosthesis is manufactured using a first mold and a second mold, the formed outer shell encloses the second mold, and the second mold is made of a soluble material and is used to form the cavity inside the outer shell. In an embodiment, the first mold is made of a Polylactic Acid (PLA) material.

In S104, the outer shell is demolded and immersed in a solution to dissolve the second mold within the outer shell, forming the cavity inside the outer shell. In an embodiment, the second mold is made of a water-soluble material, such as polyvinyl alcohol (PVA). After demolding the outer shell, the outer shell is immersed in water to dissolve the internal PVA mold, thereby creating the desired internal cavity.

In S106, the opening is formed on the outer shell for inflating and deflating the cavity.

In S108, the airbag inner layer is manufactured. A material of the airbag inner layer can be a material of an airbag, such as thermoplastic polyurethane (TPU) or thermoplastic elastomers.

In S110, the gas valve is installed on the airbag inner layer. The gas valve can be a one-way gas valve or a two-way gas valve.

In S112, the airbag inner layer is placed into the cavity of the outer shell through the opening, with the gas valve located at the opening of the outer shell. When the airbag inner layer is placed into the cavity of the outer shell through the opening, the gas valve is located at the opening of the outer shell.

In the above embodiment, a soluble mold is used to form the internal cavity of the outer shell, and the airbag inner layer is placed into the cavity of the outer shell. The airbag is designed as the inner layer, which fits the inner wall of the outer shell (e.g., the silicone outer layer), while also ensuring the leak-proof integrity.

Different implementations of the first mold and the second mold are introduced below.

The second mold can be made of various materials, such as water-soluble, oil-soluble, or alcohol-soluble materials. In an embodiment, the second mold is made of a water-soluble material, such as PVA, Biodgradable Vinyl Alcohol (BVOH), or Polyvinyl Alcohol (PVOH), which is suitable for use with the first mold made of a material such as PLA or Polyethylene Terephthalate Glycol-Modified (PETG), and water is used as the solvent. This implementation is more effective when a supporting structure is complex, and the material is environmentally friendly and easily soluble.

In an embodiment, the second mold is made of an oil-soluble material, such as High Impact Polystyrene (HIPS), which is suitable for use with the first mold made of a high-temperature material such as Acrylonitrile Butadiene Styrene (ABS), and limonene can be used as a solvent. This implementation is more suitable for processing in high-temperature environments.

In an embodiment, the second mold is made of a chemical solvent-dissolving material, such as Polyphenylene Sulfide Resin (PPSF), which is suitable for industrial applications, resistant to high temperatures and chemical corrosion, and soluble in organic solvents.

Water and some organic solvents (polar organic solvents such as water, ethanol, isopropanol, propylene glycol, and glycerol), such as Polyethylene Glycol (PEG), have good biocompatibility and are suitable for medical 3D printing and applications that require compatibility with biomaterials.

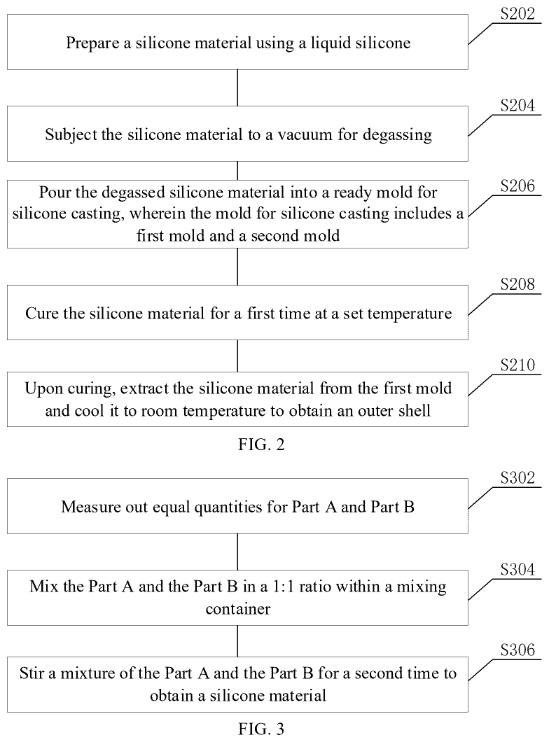

FIG. 2 shows a flowchart of a method for manufacturing a silicone outer shell according to an embodiment of the present disclosure

As shown in FIG. 2, in S202, the silicone material is prepared using a liquid silicone.

In S204, the silicone material is subject to a vacuum for degassing to eliminate any entrapped air in the silicone material. In an embodiment, the silicone material is subject to the vacuum for 5 minutes or more, preferably 5 to 20 minutes, for example, 5, 10, 15, or 20 minutes.

In S206, the degassed silicone material is poured into a ready mold for silicone casting, and the mold for silicone casting includes the first mold and the second mold.

In S208, the silicone material is cured at a set temperature for a first time. In an embodiment, the set temperature is 50 degrees Celsius. In an embodiment, the first time is 5 hours or more, preferably 12 to 16 hours, for example, 12, 15, or 16 hours.

In S210, after the silicone material is cured, the outer shell is extracted from the first mold and cooled to room temperature to obtain the outer shell. At this point, the outer shell contains the second mold. The outer shell is placed in a solution to dissolve the second mold to obtain the cavity of the outer shell.

In the above embodiment, a specific design of the outer shell and a distinctive method for manufacturing the outer shell are provided. The outer shell that meets the requirements is obtained through a prescribed process flow, which is easy to implement and environmentally friendly.

In an embodiment, the complete curing process includes the following steps:
1. curing at room temperature for 5 hours.
2. exposing the silicone to 80 degrees Celsius for 2 hours.
3. further exposing the silicone to 100 degrees Celsius for 1 hour.

After curing for 5 hours at the room temperature, parts can be handled but are not fully cured. After the above steps 2 and 3 are completed, optimum physical and performance properties are achieved.

FIG. 3 shows a flowchart of a method for preparing a silicone material according to an embodiment of the present disclosure. In this embodiment, the liquid silicone (e.g., Smooth-On Dragon Skin 10 medium, which is a silicone material) can be for example used to prepare the silicone material.

As shown in FIG. 3, in S302, equal quantities for both Part A and Part B are measured out, for example, equal quantities of 70 grams for both Part A and Part B are measured out, respectively.

In S304, the Part A and the Part B are mixed in a 1:1 ratio within a mixing container.

In S306, a mixture of the Part A and the Part B is stirred for a second time to make the mixture homogeneous by stirring to obtain the silicone material. In an embodiment, the mixture is stirred vigorously for 3 minutes, with attention paid to the container's sides and bottom to ensure a homogeneous mixture.

In the above embodiment, the silicone material that meets the requirements is prepared through accurate operations.

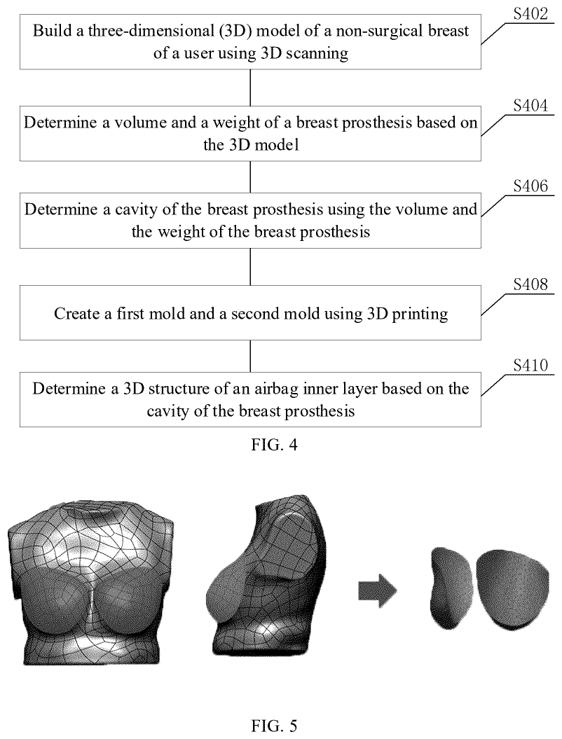

FIG. 4 shows a flowchart of a method for determining a mold using 3D scanning according to an embodiment of the present disclosure.

As shown in FIG. 4, in S402, a three-dimensional (3D) model of a non-surgical breast of a user (a patient) is built using 3D scanning for volume and weight calculations.

In S404, a volume and a weight of the breast prosthesis are determined based on the obtained 3D model. With the 3D scanned model, the breast can be separated from the chest wall using Boolean operations (see FIG. 5), and the volume can be calculated according to it. In an embodiment, the average density from the literature and a formula (mass m=1/3ρV, where V is the breast volume and ρ is the uniform breast density) are used to calculate the breast weight. The volume and weight of the breast prosthesis are consistent with those of the non-surgical breast.

In S406, the volume of the cavity of the breast prosthesis is determined using the volume and the weight of the breast prosthesis. In an embodiment, the shape and weight of the silicone outer shell match the shape and weight of the non-surgically altered real breast. Using the density of silicone (e.g., 1.1 mg/m³), the volume of the cavity can be calculated, thereby determining a design that meets the requirements.

In S408, the first mold and the second mold are created by 3D printing.

In S410, a 3D structure of the airbag inner layer is determined based on the cavity of the breast prosthesis.

In the above embodiment, the design of the breast prosthesis aims to ensure that it matches the shape and weight of the real breast without surgery. The design of the breast prosthesis, featuring the silicone outer layer and the TPU airbag inner layer, utilizes 3D scanning and 3D printing technologies, which ensures that the prosthesis matches the shape and weight of the patient's remaining natural breast, providing symmetry of the body.

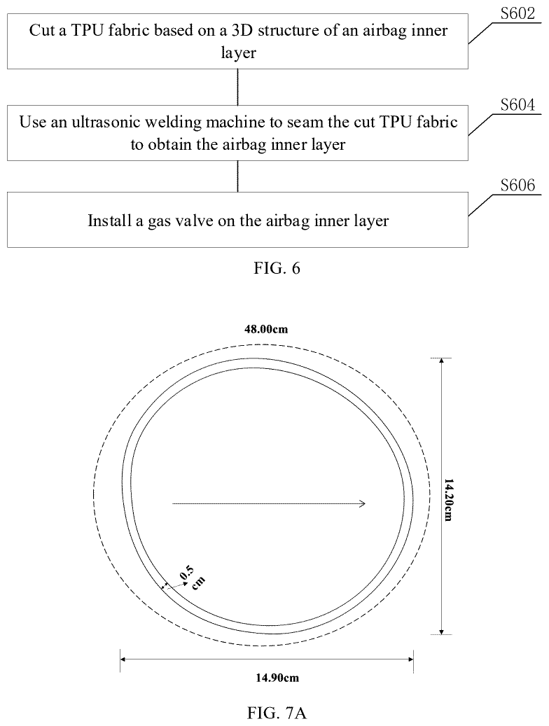

FIG. 6 shows a flowchart of a method for manufacturing an airbag inner layer according to an embodiment of the present disclosure.

As shown in FIG. 6, in S602, a TPU fabric is cut based on the 3D structure of the airbag inner layer.

In S604, an ultrasonic welding machine is used to seam the cut TPU fabric to obtain the airbag inner layer.

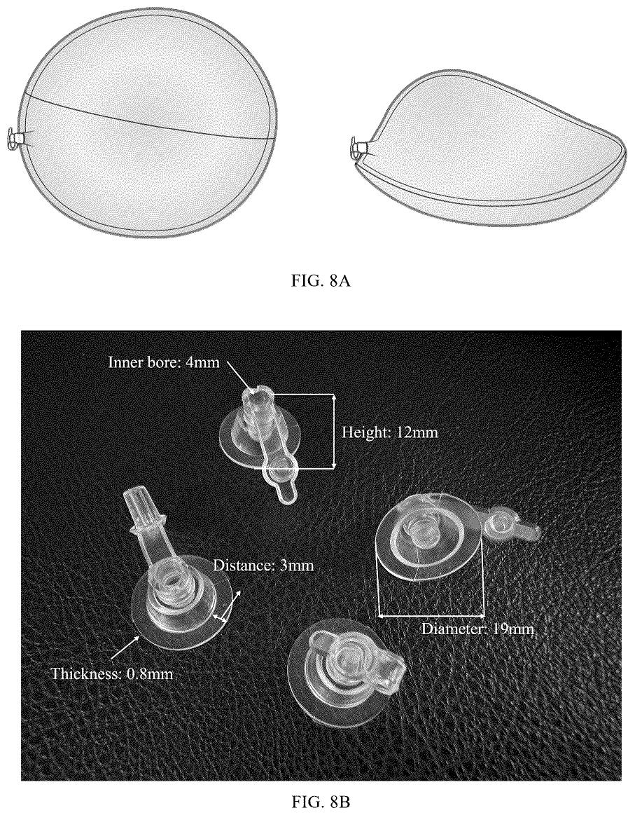

In S606, the gas valve is installed on the airbag inner layer. In an embodiment, a one-way gas valve is installed on the airbag inner layer (see FIG. 8), which allows air intake but prevents automatic air release, thereby effectively maintaining the shape of the airbag.

In the above embodiment, the airbag is utilized to reduce the volume of silicone, thereby reducing the weight. Additionally, by inflating and deflating, the volume can be adjusted within a certain range. Combining 3D scanning and 3D printing technologies, the non-surgical breast is matched, thereby resolving the issue of the breast prosthesis not fitting the body properly.

Through the above method, the main design of the pneumatic breast prosthesis is realized.

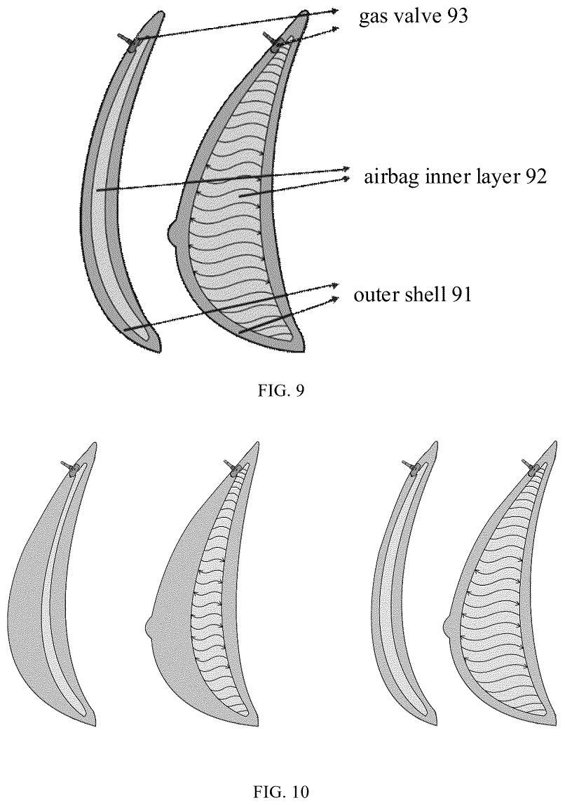

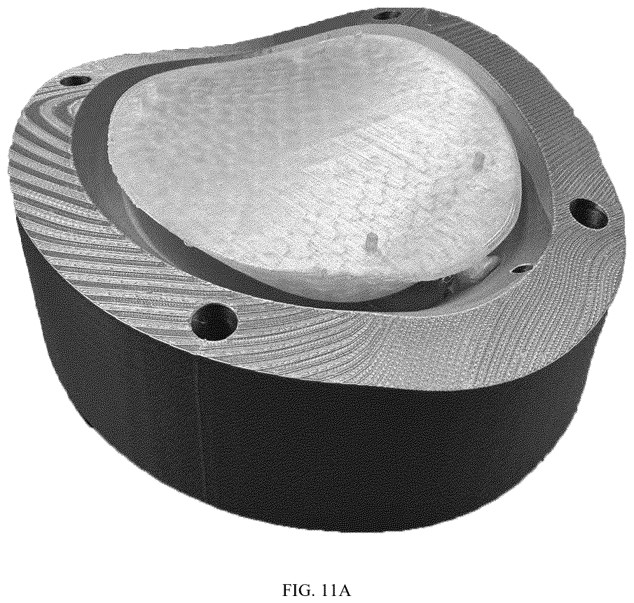

Referring to FIG. 9, the breast prosthesis consists primarily of two parts: a cover layer (or referred as to the outer shell) 91, such as a silicone outer shell, and an airbag inner layer 92. The airbag inner layer has a gas valve 93 located at the opening of the silicone cover layer. Silicone is used as the cover layer to provide a natural feel and to contribute to most of the weight of the breast prosthesis. The weight of silicone is adjusted according to the weight of the natural breast, resulting in varying volumes of silicone among individuals (see FIG. 10). The airbag is used as the inner layer to reduce the weight and allow for shape adjustment. This design is suitable for individuals with different cup sizes, accommodating body weight changes, and compatible with various underwear styles. The airbag is made of thermoplastic polyurethane (TPU), which is thermoplastic elastomers with many properties, including elasticity, transparency, and resistance to oil, grease, and abrasion. The impermeability of TPU makes it suitable as a material for the airbag, and by utilizing its thermoplastic properties, it can be conveniently cut and seamed using the ultrasonic welding machine.

The following is a specific application example. The production process is as follows.
1. For the silicon outer layer, the 3D scanning technology is used to build a 3D model of the non-surgical real breast for volume and weight calculations. FIG. 5 shows a breast model extracted from a scanned model according to an embodiment of the present disclosure. With the scanned model, the breast can be separated from the chest wall using Boolean operations, and the volume can be calculated according to it. The average density from the literature and a formula (mass m=1/3ρV, where V is the breast volume and ρ is the uniform breast density) are used to calculate the breast weight.

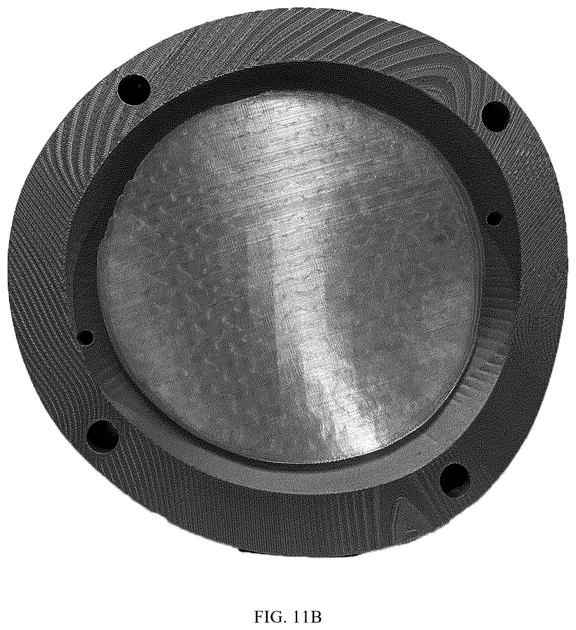

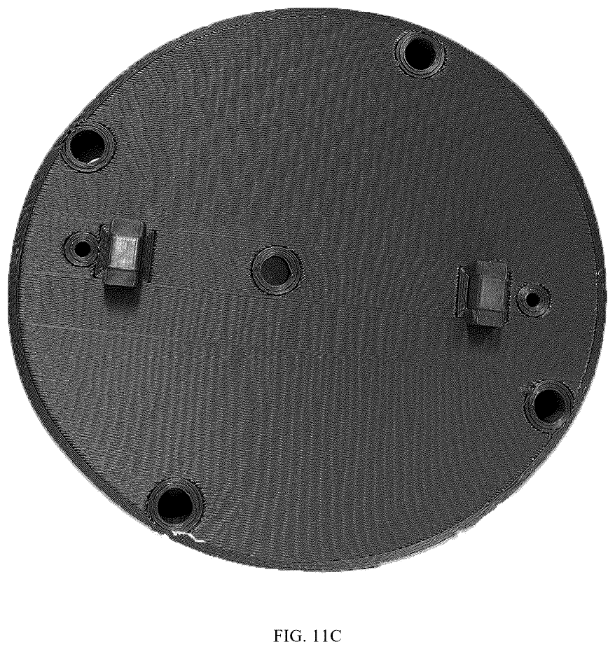

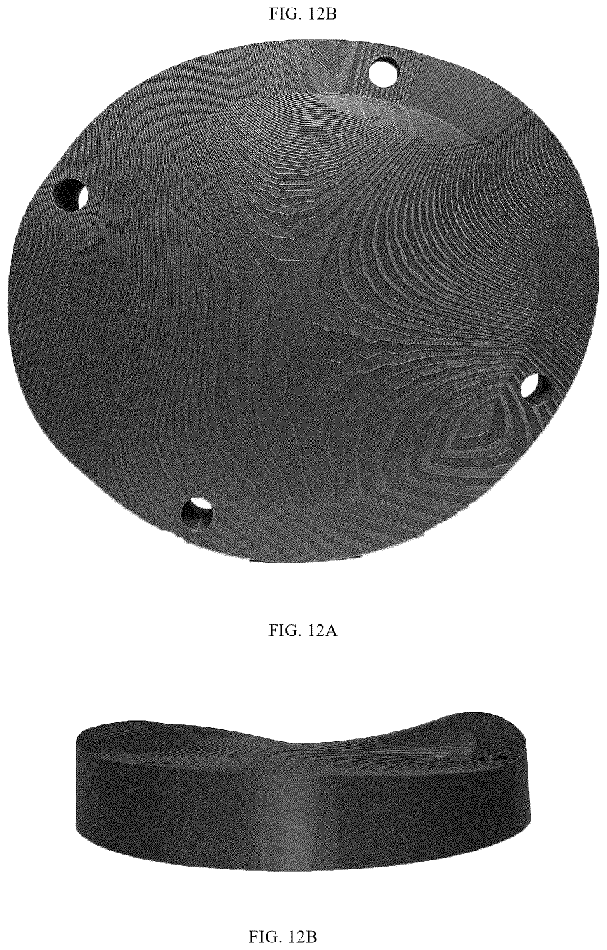

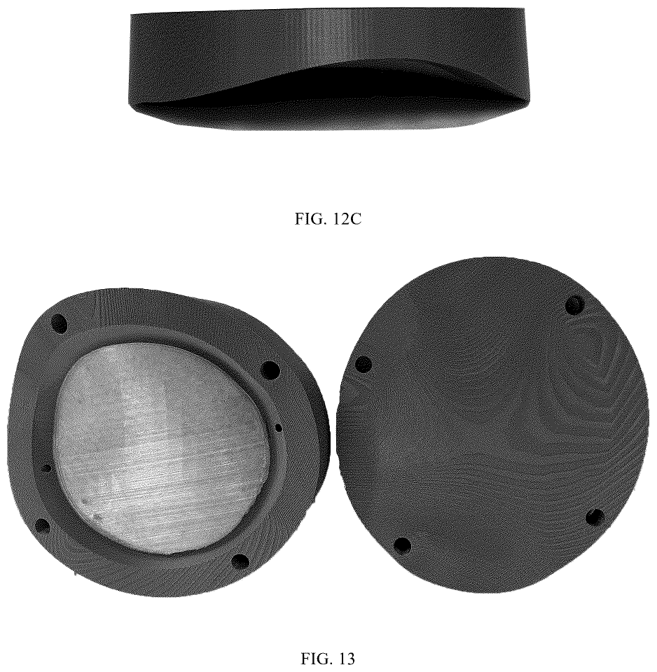

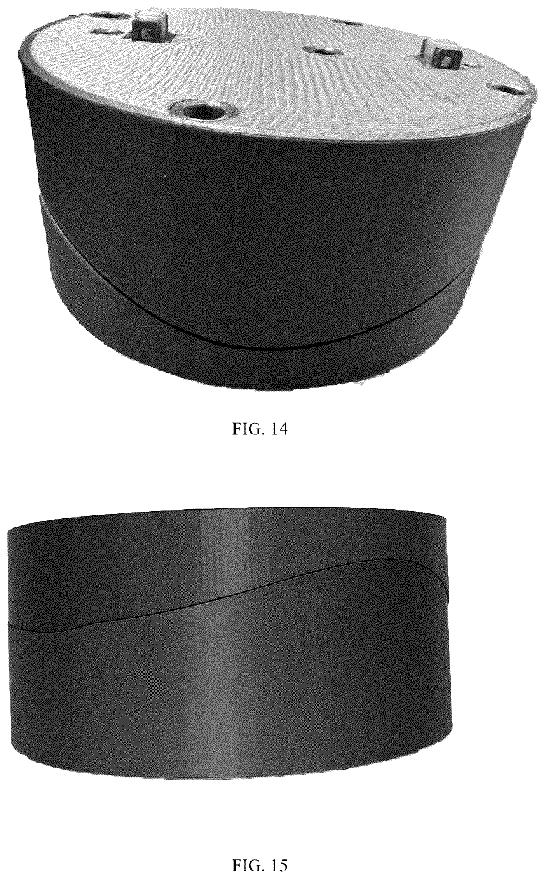

The shape and weight of the silicone outer layer should match those of the non-surgically altered real breast. Using the density (1.1 Mg/m³), the volume can be calculated, allowing for the design of the outer layer. Then, the 3D printing technology is utilized to produce a casting mold for this silicone outer layer. Molds for silicone casting are created using the 3D printing technology. The outer mold (also called the first mold) uses, for example, PLA, while the inner mold (also called the second mold) uses a soluble material (e.g., a water-soluble material), which can be effortlessly dissolved post-curing. FIGS. 11A to 11C show diagrams of a first portion of a first mold and a second mold at different angles according to an embodiment of the present disclosure. As shown in FIG. 11A, the mold for silicone casting includes an externally positioned first mold (which is specifically the first portion of the first mold) and a second mold. The second mold has a support portion that contacts the first mold to form a gap between the first and second molds, in order to form the outer shell. In an embodiment, dissolution of the support portion of the second mold creates the opening in the outer shell. FIGS. 12A to 12C show diagrams of a second portion of a first mold at different angles according to an embodiment of the present disclosure. FIG. 13 shows top views of a first portion of a first mold, a second mold, and a second portion of the first mold according to an embodiment of the present disclosure. FIG. 14 shows a perspective view of a first mold and a second mold after being combined according to an embodiment of the present disclosure. As shown in FIG. 14, the first portion of the first mold has a hole for injecting a material forming the outer shell, such as silicone. A raised portion in FIG. 14 is used to fix the mold. FIG. 15 shows a side view of a first mold and a second mold after being combined according to an embodiment of the present disclosure.

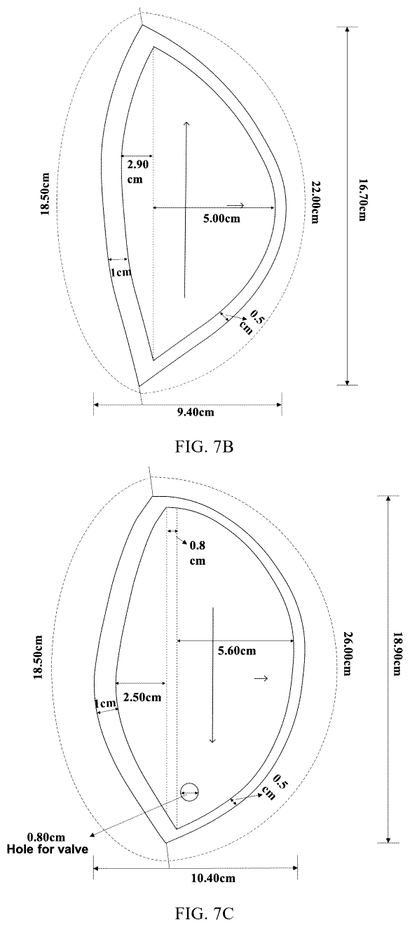

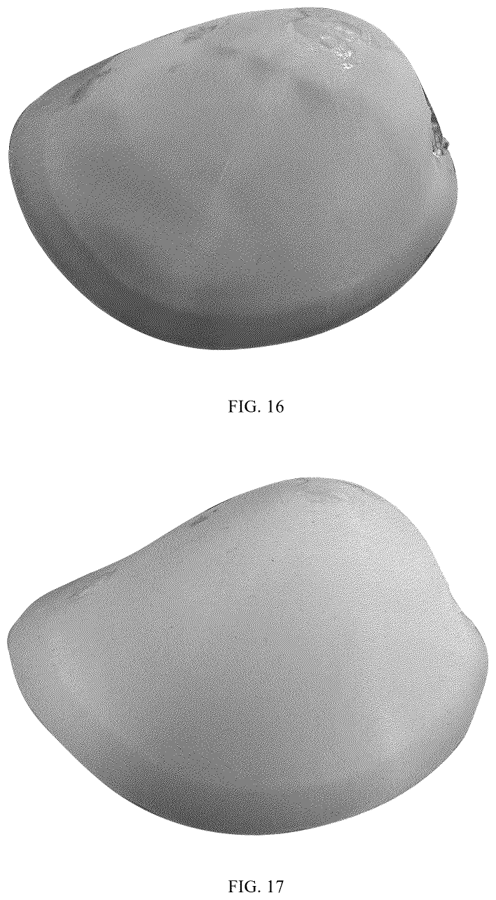

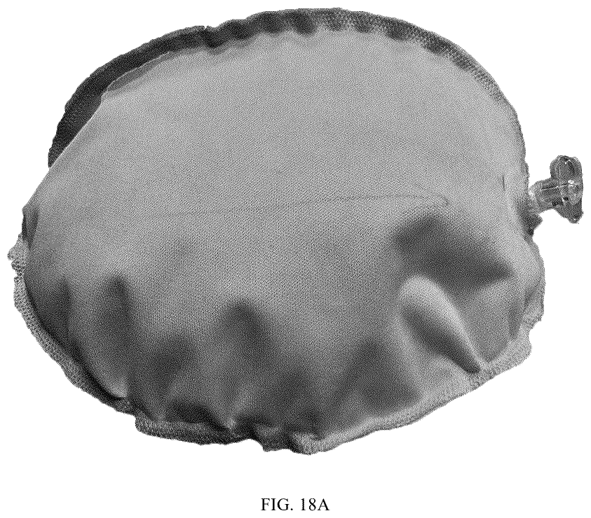

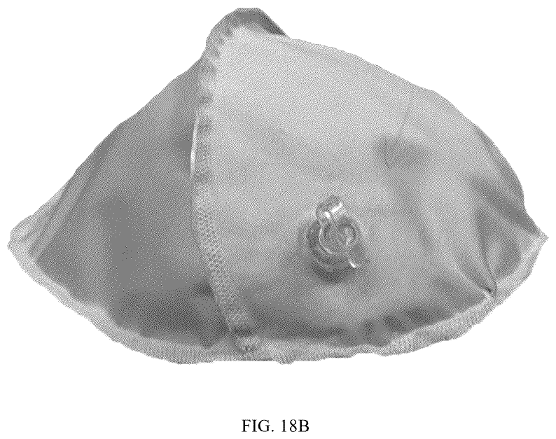

The next step is the preparation of the silicone material using the liquid silicone, such as Smooth-On Dragon Skin 10 Medium. Equal quantities of 70 grams for both Part A and Part B are measured out. These components are mixed in a 1:1 ratio within a mixing container. The mixture is stirred vigorously for 3 minutes, with attention paid to the container's sides and bottom to ensure a homogeneous mixture. To eliminate any entrapped air, the mixture is subjected to a vacuum for 15 minutes, a procedure known as de-airing. The degassed silicone is then carefully poured into the ready 3D printed mold (e.g., the mold shown in FIG. 14, where the silicone is injected through holes in the mold), with meticulous attention to prevent the introduction of bubbles or imperfections. The silicone is left to cure at a controlled room temperature of approximately 50° C. for a duration of 10-15 hours. Upon curing, the silicone is extracted from the mold and allowed to return to room temperature, and the silicone form is demoulded, obtaining the outer shell. FIG. 16 shows a schematic diagram of a silicone outer shell after demoulding according to an embodiment of the present disclosure. The obtained outer shell is immersed in water to dissolve the internal PVA mold, thus creating the desired internal cavity of the outer shell. FIG. 17 shows a schematic diagram of a silicone outer shell after forming an internal cavity according to an embodiment of the present disclosure. After completion, a small hole is opened on the upper side of the outer body for the purpose of inflation and deflation.
2. In the production of the airbag, to ensure a closer fit with the shape of the outer layer silicone, a 3D structure is also designed. Its pattern is shown in FIGS. 7A to 7C, which provide a prototype of the custom-fit pneumatic breast prosthesis for a cup size 38C, where specific dimensions for different parts are shown. FIG. 7A shows a bottom schematic diagram of a design pattern of an airbag (an example of a cup size 38C) according to an embodiment of the present disclosure, FIG. 7B shows a first side schematic diagram of a design pattern of an airbag (an example of a cup size 38C) according to an embodiment of the present disclosure, and FIG. 7C shows a second side schematic diagram of a design pattern of an airbag (an example of a cup size 38C) according to an embodiment of the present disclosure. The TPU fabric is cut, the ultrasonic welding machine is used for seaming, and the one-way gas valve is installed, which allows air intake but prevents automatic air release, thereby effectively maintaining the shape of the airbag. FIG. 8A shows a schematic perspective view of a design of an airbag inner layer according to an embodiment of the present disclosure, and FIG. 8B shows a schematic view of a one-way gas valve according to an embodiment of the present disclosure. FIGS. 18A to 18B show schematic diagrams of an airbag inner layer at different angles according to an embodiment of the present disclosure.

By producing various sizes of prototypes and conducting wear trials with each subject, any deficiency in the design and any discomfort experienced while wearing the product are identified.

In embodiments of the present disclosure, the issue of post-mastectomy body asymmetry is addressed by providing solutions for both aesthetic appearance and weight distribution. The design of the airbag reduces the weight of the prosthesis, solving the issue of discomfort caused by excessive heaviness. Combining 3D scanning and 3D printing technologies, the invention matches the unsurgical breast and resolves the issue of the prosthesis not fitting the body properly.

It is ensured the same shape and weight as the unsurgical breast, guaranteeing body symmetry and comfort

In embodiments of the present disclosure, two methods for installing a breast prosthesis are provided: it can be placed into a bra pocket or attached directly to the skin, ensuring compatibility with most outfits.

In addition, although the various steps of the method of the present disclosure are described in a particular order in the figures, this is not required or implied that the steps must be performed in the specific order, or all the steps shown must be performed to achieve the desired result. Additionally or alternatively, certain steps may be omitted, multiple steps may be combined into one step, and/or one step may be decomposed into multiple steps and so on.

Other embodiments of the present disclosure will be apparent to those skilled in the art from consideration of the specification and practice of the present disclosure disclosed herein. The present application is intended to cover any variations, uses, or adaptations of the present disclosure, which are in accordance with the general principles of the present disclosure and include common general knowledge or conventional technical means in the art that are not disclosed in the present disclosure. The specification and embodiments are illustrative, and the real scope and spirit of the present disclosure is defined by the appended claims.

What is claimed is:

1. A method for manufacturing a breast prosthesis, wherein the breast prosthesis comprises an outer shell and an airbag inner layer, a cavity is formed inside the outer shell, the outer shell is provided with an opening that is in communication with the cavity, and the airbag inner layer is provided with a gas valve, and wherein the method comprises:
manufacturing the outer shell using a first mold and a second mold, wherein the second mold is enclosed by the outer shell, and the second mold is made of a soluble material;
demolding the outer shell and immersing the outer shell in a solution to dissolve the second mold within the outer shell to form the cavity inside the outer shell;
forming the opening in the outer shell;
manufacturing the airbag inner layer; and
installing the gas valve on the airbag inner layer to locate the gas valve at the opening of the outer shell upon placing the airbag inner layer into the cavity of the outer shell through the opening.

2. The method according to claim 1, wherein the outer shell is made of silicone; and
manufacturing the outer shell using the first mold and the second mold comprises:
preparing a silicone material using a liquid silicone;
subjecting the silicone material to a vacuum for degassing;
pouring the degassed silicone material into a ready mold for silicone casting, wherein the mold for silicone casting comprises the first mold and the second mold;
curing the silicone material for a first time at a set temperature; and
upon curing, extracting the outer shell from the first mold and cooling the outer shell to room temperature.

3. The method according to claim 2, wherein preparing the silicone material using the liquid silicone comprises:
measuring out equal quantities for Part A and Part B;
mixing the Part A and the Part B in a 1:1 ratio within a mixing container; and
stirring a mixture of the Part A and the Part B for a second time to obtain the silicone material.

4. The method according to claim 3, wherein the set temperature is 50° C., the first time is between 12 and 16 hours, and the second time is between 3 and 5 minutes; and
subjecting the silicone material to the vacuum for degassing comprises:
subjecting the silicone material to the vacuum for 5 to 15 minutes for degassing.

5. The method according to claim 1, further comprising:
building a three-dimensional (3D) model of a non-surgical breast of a user using 3D scanning;
determining a volume and a weight of the breast prosthesis based on the 3D model;
determining the cavity of the breast prosthesis using the volume and the weight of the breast prosthesis; and
creating the first mold and the second mold using 3D printing.

6. The method according to claim 5, wherein the airbag inner layer is made of thermoplastic polyurethane (TPU); and
the method further comprises:
determining a 3D structure of the airbag inner layer based on the cavity of the breast prosthesis to make the airbag inner layer fit an inner wall of the cavity of the outer shell;
cutting a TPU fabric based on the 3D structure of the airbag inner layer;
using an ultrasonic welding machine for seaming to obtain the airbag inner layer; and
installing the gas valve on the airbag inner layer.

7. The method according to claim 1, wherein the gas valve is a one-way gas valve.

8. The method according to claim 1, wherein the outer shell is made of silicone, a polymer material, or a biocompatible material.

9. The method according to claim 1, wherein the airbag inner layer is made of thermoplastic polyurethane (TPU); and/or
the second mold is made of water-soluble material, an oil-soluble material, or an alcohol-soluble material.

10. The method according to claim 1, wherein the second mold is made of water-soluble material, an oil-soluble material, or an alcohol-soluble material.

11. A breast prosthesis, comprising:
an outer shell, wherein a cavity is formed inside the outer shell, and the outer shell is provided with an opening that is in communication with the cavity; and
an airbag inner layer located within the cavity, wherein the airbag inner layer is provided with a gas valve for filling the airbag inner layer with gas.

12. The breast prosthesis according to claim 11, wherein the gas valve is a one-way gas valve.

13. The breast prosthesis according to claim 11, wherein the outer shell is made of silicone, a polymer material or a biocompatible material.

14. The breast prosthesis according to claim 11, wherein the airbag inner layer is made of thermoplastic polyurethane.
